(19) United States
(12) Patent Application Publication
(10) Pub. No.: US 2026/0137390 A1
(43) Pub. Date: May 21, 2026

(54) SYSTEMS AND METHODS FOR DISPENSING EMBOLIC PARTICLES FOR EMBOLIZATION

(71) Applicants: STRYKER CORPORATION, Portage, MI (US); STRYKER EUROPEAN OPERATIONS LIMITED, Carrigtwohill (IE)

(72) Inventors: Stephen Porter, Piedmont, CA (US); Han-Wei Wang, Fremont, CA (US)

(73) Assignees: STRYKER CORPORATION, Portage, MI (US); STRYKER EUROPEAN OPERATIONS LIMITED, Carrigtwohill (IE)

(21) Appl. No.: 19/450,749

(22) Filed: Jan. 16, 2026

Related U.S. Application Data
(63) Continuation of application No. PCT/US2024/045887, filed on Sep. 9, 2024.
(60) Provisional application No. 63/583,991, filed on Sep. 20, 2023.

(51) Int. Cl. A61B 17/12

(52) U.S. Cl. CPC A61B 17/12186; A61B 17/12109; A61B 2017/1205

(57) ABSTRACT

Systems and methods for performing embolization procedures for treating various medical indications and targets such as hemorrhaging, tumors utilize a mixture of embolic particles and delivery media at a very low concentration of embolic materials to delivery media, while intermittently injecting a bolus of contrast media for visualizing the progress of the embolization procedure. The systems and methods are also automated to minimize the user interaction and may also incorporate sensors and feedback control processes to control the systems and methods in performing an embolization procedure.

### RELATED APPLICATION DATA

The application is a continuation of International Patent Application No. PCT/US2024/045887, filed on Sep. 9, 2024, which claims the benefit of U.S. Provisional Patent Application Ser. No. 63/583,991, filed on Sep. 20, 2023, the entire disclosures of all of which are hereby incorporated herein by reference in their entirety into the present application.

### FIELD

The field of the disclosure generally relates to medical systems and methods, and more particularly, to systems and methods for dispensing embolic particles and contrast media for efficient embolization of blood vessels in target tissue of a patient.

### BACKGROUND

Embolization of blood vessels, such as arteries, using embolic micro-particles is a minimally invasive interventional radiology procedure used to treat various medical conditions. It involves the selective occlusion of blood vessels by injecting tiny particles, such as polymer foam fragments or beads, into the arteries via an intravascular catheter, thereby obstructing the blood flow to a specific area. This technique has been shown to be effective in managing bleeding, shrinking tumors, and treating certain vascular malformations.

Typically, the procedure begins with the patient lying on an examination table in an angiography suite. After applying local anesthesia, a small incision is made near an accessible artery, such as in the groin area to access the femoral artery. A catheter is inserted into the artery and guided through the blood vessels using fluoroscopic imaging, which provides real-time X-ray guidance.

The catheter is carefully navigated to the target blood vessels, often using a combination of angiography and roadmapping techniques. These imaging methods help the interventional radiologist visualize the vascular anatomy and identify the vessels leading to the site of interest, such as target arteries and/or capillaries in the area of target tissue to be treated by embolization. For example, the target arteries and/or capillaries to be embolized may transport blood to a target tissue which is hemorrhaging or has a tumor, fibroids, a vascular malformation or other condition to be treated.

Once the catheter is positioned proximate the target artery, a contrast agent is injected through the catheter to confirm the precise location and evaluate the blood flow. This assists in assessing and planning the injection of micro-particles to ensure that the micro-particles will be delivered precisely to the intended area.

After confirming the optimal position, a syringe filled with a mixture of embolic micro-particles and contrast agent is used to inject the mixture into the catheter. The embolic particles are commonly composed of biocompatible materials like gelatin sponge or polyvinyl alcohol (PVA), and may be tiny spheres or other shapes that can range in size from 50 to 1,000 micrometers. The size of the particles used depends on the specific condition being treated and the size of the catheter being utilized. The mixture of embolic micro-particles and contrast agent flows through the catheter in the vascular system to the target location. The mixture of embolic particles and contrast agent are ejected from the distal end of catheter and flow through the vascular system, and the embolic particles eventually reach and lodge into smaller blood vessels that supply the tissue area being targeted.

The practitioner visualizes the progress of the embolic particle/contrast mixture, using the contrast agent. In order to visualize the flow of the embolic particle/contrast mixture and extent of flow reduction in the target blood vessels, short “puffs” (i.e., a high concentration burst of embolic particle/contrast mixture) are intermittently injected through the catheter. Without these “puffs,” the injected embolic particle/contrast mixture simply appears as substantially uniform gray lines along the flow path. In contrast, the “puffs” allow for effective visualization of the speed of flow and depth of perfusion indicating the extent of the flow reduction in the blood flow field, and monitoring of diminishing washout time of the contrast “puffs” and reduction of flow penetration shows the progression of embolization.

However, the relatively high concentration bursts (“puffs”) of embolic particle/contrast mixture in a relatively high viscosity medium are prone to create clogging events in undesired locations in the blood vessels, or undesirable proximal vessel aggregation. As a result, the extent of distal penetration and uniformity of embolization is adversely affected. In addition, the high-flow, high-viscosity injections are also much more prone to reflux, which is the backward flow of embolic material or particles during the procedure. Reflux can occur when the embolic material travels downstream and then partially or completely reverses its course due to high pressure and/or overly sufficient occlusion. This is undesirable because it leads to unintended embolization in other areas or reduced effectiveness of the embolization.

As the embolic particles accumulate, they block or embolize the blood vessels obstructing the blood flow, thereby causing ischemia (lack of blood supply) to the target tissue region. The embolization of the blood vessels controls bleeding in the case of treating hemorrhaging or leads to tumor shrinkage by depriving the target tissue of nutrients and oxygen when treating tumorous tissue.

After the injection is complete, the catheter is removed, and the incision is closed to prevent bleeding. The patient is then monitored for a few hours as an out-patient procedure or overnight to ensure there are no immediate complications.

Arterial embolization using micro-particles offers several advantages over traditional surgical interventions. It is less invasive, resulting in smaller incisions, reduced pain, and shorter hospital stays. The targeted approach minimizes damage to healthy tissues, making it an effective treatment option for both malignant and benign conditions.

The specific outcomes of arterial embolization vary depending on the underlying condition being treated. In cases of bleeding, the procedure can rapidly stop the hemorrhage and stabilize the patient. For tumors, embolization can lead to tumor shrinkage, reducing symptoms and facilitating subsequent surgical resection. Arterial embolization using micro-particles is a valuable interventional radiology technique for managing bleeding, shrinking tumors, controlling angiogenetic inflammation, and treating vascular malformations. By selectively occluding blood vessels, this minimally invasive procedure provides an effective alternative to traditional surgery, offering patients reduced risks, quicker recovery times, and improved outcomes.

Still, there remains a need for improved devices, systems and methods for performing arterial embolization which overcome the drawbacks and deficiencies of previous arterial embolization techniques.

### SUMMARY

Disclosed herein are innovative systems and methods for performing embolization procedures for treating various medical indications and targets such as hemorrhaging, tumors, fibroids, prostate, thyroid, geniculate artery, etc. In one highly innovative aspect, the systems and methods are configured to inject a mixture of embolic particles and delivery media at a very low concentration of embolic materials to delivery media, while intermittently injecting a bolus of contrast media for visualizing the progress of the embolization procedure.

According to one disclosed embodiment, an embolization system comprises a first dispenser for dispensing an embolic particle mixture into a catheter. As some examples, the first dispenser may be a syringe, a pressure chamber connected to a pressure source or gravity fed dispenser with a valve for controlling the dispensing, a metering pump or any other suitable dispenser capable of controlling the dispensing of a fluid at a specified pressure, flow rate or bolus volume. The embolic particle mixture comprises a mixture of embolic particles and delivery media. In various aspects, the delivery media may be saline, other compatible fluid for carrying the embolic particle. The first dispenser is electronically controllable to controllably dispense the embolic particle mixture.

The embolization system also includes a second dispenser for dispensing a contrast media into the catheter. The second dispenser may be any of the dispenser types described for the first dispenser, and may be of the same type or a different type.

The first dispenser and second dispenser are each connected to a manifold which is in fluid communication with both the first dispenser and the second dispenser such that the first dispenser and second dispenser dispense into the manifold. The manifold has a dispensing outlet for dispensing out of the manifold and into the catheter. Accordingly, the embolization system is capable of switching between injection of two distinctly different fluid mixtures having two different purposes, namely an embolic particle mixture without contrast media for embolizing a target tissue region, and a contrast media mixture comprising contrast media for visualizing the embolization. The embolization system can dispense the two different fluid mixtures differentially from each other in a repeating pattern. In additional aspects, the embolization system is also configured to automatically control (e.g., controlling the injection rates) the first dispenser in dispensing embolic particle mixture and the second dispenser in dispensing contrast media. As used herein, the term “automatically,” “automatic,” and/or other forms with respect to controlling a dispensing/injection function means that the flow rate, duration and/or volume of fluid dispensing are controlled without human action or intervention, but allows for manual triggering or automated triggering of an “automatic” dispensing. For example, an automated injection (or automated dispensing) may be an injection at a flow rate and a total volume of fluid controlled by a controller of the system which is initiated by a user triggering the injection manually such as by pushing a button on the controller.

In another aspect, the first dispenser dispenses the embolic particle mixture at low concentration in a relatively large volume. This avoids using a significant concentration of contrast media, as in prior art systems and methods which dispense a mixture of embolic particles and contrast media. Using large total volumes of contrast media is ill advised because it is important to limit the amount of contrast media injected into a patient to prevent kidney damage. This also allows the second dispenser to separately dispense a high concentration of contrast without embolic particles in low volume to allow for good visualization of blood flow (i.e., good visualization of the progress of the embolization procedure as blood flow is blocked by the embolization). Furthermore, the contrast media has a higher viscosity than the embolic particle mixture, and therefor is more likely to show stasis or reflux within the arteries.

In another aspect, the embolic particle mixture has a specific low concentration of embolic particles to injected embolic particle mixture. For example, the low concentration of embolic particles to embolic particle mixture may be from 2.0%-0.05% by volume, or from 1.0-0.1% by volume. This is in the case of a total volume of embolic particle mixture injected in the range of from 50-500 cc (cubic cm); a volume of media contrast diluted with saline injected in the range of 10-50 cc; a cumulative time of injection of embolic particle mixture in the range of 5-20 minutes; and a flow rate of injection of embolic particle mixture in the range of 2.0-0.1 cc/second. Thus, the low concentration of embolic particles to embolic particle mixture may be suitably adjusted where these embolization characteristics are varied from these ranges. The size of the embolic particles may also affect the low concentration embolic particles to embolic particle mixture.

In another aspect of the embolization system, the first dispenser and second dispenser each comprise a powered syringe driven by an actuator configured to controllably dispense a pressurized flow of fluid. As used herein, the term “powered” means that a device is not manually driven, and includes without limitation, driven electrically, pneumatically, hydraulically, etc. For example, the powered syringe may be a motorized syringe powered by an electric motor, and the electric motorized syringes may be controllable by a motor controller configured to control the speed at which the plunger of the syringe is actuated to thereby control the flow rate and/or pressure of dispensing by the respective dispenser.

In another aspect of the system, the first dispenser may separately dispense the embolic particles and the delivery media and mix them to prepare and dispense the embolic particle mixture. For example, the first dispenser may comprise a first sub-dispenser for dispensing the embolic particles into a mixer and a second sub-dispenser for dispensing the delivery media into the mixer. The system may further comprise a mixer in fluid communication with the first sub-dispenser and the second sub-dispenser. The mixer is configured to mix the embolic particles and the delivery media and output the well-mixed embolic particle mixture. In additional aspects, the mixer may be a passive mixer such as a flow induced mixer, static mixer, vortex mixer, an active mixer such as a mixer having a moving mixing element, or an integrated mixer with acoustic stirring or piezo-electric transducers. In another aspect, the first sub-dispenser and second sub-dispenser may comprise motorized syringes, same or similar to the motorized syringes described herein.

In yet another aspect, the embolization system may include a controller operably coupled to the first dispenser and the second dispenser. The controller is configured to automatically control the first dispenser and the second dispenser to controllably dispense the embolic mixture from the first dispenser and controllably dispense the contrast media from the second dispenser. For example, the controller may have a computer processor or other electronic control logic configured to electronically control the operation of the first dispenser and second dispenser. In still another aspect, the controller may be selectably programmable by the user to control a characteristic of the dispensing of the embolic particle mixture by the first dispenser and the dispensing of the contrast media by the second dispenser. For example, the controller may be configured to allow a user to set one or more of the following characteristics of dispensing by the first and second dispensers: (1) a flow rate of dispensing, (2) a pressure of dispensing, (3) frequency of dispensing contrast media, (4) actuation of an active mixing, etc.

In still another aspect, the embolization system may include one or more sensors for monitoring and/or controlling the operation of the embolization system. In one aspect, the system may further comprise a first sensor operably connected to the controller and configured to detect an attribute of the dispensing by at least one of the first dispenser and second dispenser and to output a first sensor signal related to the attribute. For example, the first sensor may be a flow rate sensor for detecting the flow rate of fluid dispensed by the first and second dispensers or a pressure sensor for detecting the pressure of the fluid dispensed by the first and second dispensers. The controller utilizes the first sensor signal in a feedback control process to control an attribute of the dispensing (e.g., flowrate, pressure, concentration of dispensed embolic particle mixture or contrast media, etc.). As an example, the controller may utilize a flow rate signal to control the operation of the first dispenser and/or second dispenser to adjust the flow rate of the dispensing to a pre-set flow rate set by the user. The first sensor may be any suitable sensor, such as a flow rate sensor, a pressure sensor, a force sensor, an actuator torque sensor, a motor current sensor, or the like.

In another aspect, the controller is configured to automatically perform an embolization process including dispensing an embolic particle mixture at a prescribed flow rate or pressure controlled by the controller; and intermittently dispensing a bolus of contrast media at a prescribed flow rate or pressure controlled by the controller.

In yet another aspect, the embolization system may also utilize controlled flow rectification and laminar flow effects to further prevent embolic material aggregation, surface adsorption, sedimentation and/or catheter clogging during an embolization procedure. To this end, the embolization system may also include a flow rectifier having an inlet and an outlet. The inlet is connected to, and in fluid communication with, the outlet of the manifold. The rectifier outlet is connectable to the proximal end of a catheter. The flow rectifier is configured to regulate the flow pattern of the embolic particle mixture as it flows through the rectifier to produce a more uniform flow of the embolic particles within the delivery media. For instance, the flow rectifier may be configured to produce a laminar flow of the embolic particle mixture wherein the embolic particles are aligned proximate the center line of the flow the embolic particle mixture at the outlet of the rectifier. In one example, the flow rectifier may comprise an inner tube having a tapered outlet coaxially disposed in an outer tube having a tapered outlet in which the inner tube outlet and outer tube outlet intersect. The embolic particles are flowed through the inner tube and through the tapered outlet which rectifies the particles into a narrow column (even a single file column) of embolic particles while delivery media is flowed through the outer tube to produce a laminar flow or a turbulence-reduced flow, at the outer tube outlet. Then, at the intersection of the two flows, the rectified embolic particles are positioned along the center line of the combined flow of the embolic particles and delivery media.

In another embodiment, an embolization system may be similar in most respects to the embolization system just described, except that system utilizes a first catheter for injecting the embolic particle mixture, and a second catheter for injecting the contrast media. Accordingly, the first catheter is connected to, and in fluid communication with, the first dispenser, and the second catheter is connected to, and in fluid communication with, the second dispenser. This can eliminate the need for a manifold to receive and distribute the fluid flows from first dispenser and second dispenser into a single catheter. The first catheter and second catheter may have various suitable configurations. In one aspect, the first catheter may be a microcatheter and the second catheter may be a support catheter, guide catheter or sheath catheter into which the microcatheter is inserted. In this configuration, the first dispenser injects the embolic particle mixture into and through the microcatheter, and the second dispenser injects the contrast media into and through an annular lumen formed between the outer catheter and the microcatheter. The first catheter and second catheter may be configured to either have a common outlet or different axially separated outlets for injecting the respective fluids into the target tissue region. Alternatively, the first catheter and second catheter may be positioned side by side, with each having a different outlet, or a common outlet at the distal ends of the respective first catheter and second catheter. This configuration of the embolization system allows the embolic particle mixture to be continuously infused (i.e., without stopping) via the first catheter and the contrast agent injected in pulses via the second catheter while continuously injecting embolic particle mixture.

The embolization system having first and second catheters may include any one or more of the other aspects and features described herein for the embolization systems.

Another embodiment disclosed herein is directed to a method form performing a vascular embolization procedure. The method includes automatically controlling a variable injection of two distinctly different fluid mixtures having two different purposes. Accordingly, the method automatically controls the variable dispensing of two distinctly different fluid mixtures having two different purposes, namely an embolic particle mixture without contrast media for embolizing a target tissue region, and a contrast media mixture comprising contrast media for visualizing the embolization. The method dispenses the two different fluid mixtures separately from each other in a controlled variable injection pattern. In additional aspects, the dispensing of the embolic particle mixture and/or the contrast media are automatically controlled by a controller.

The method includes injecting an embolic particle mixture via a catheter to the target region of tissue, the injecting of the embolic particle mixture is automatically controlled by a controller. A bolus of contrast media is intermittently injected via the catheter to the target region of tissue to monitor the progress of embolization of the target region of tissue on a visualization system. The bolus of contrast media may be injected manually, such as by a user manually actuating a syringe filled with contrast media. An advantage of manual contrast injection (or manually triggered-automated injection) is that the user can coordinate the contrast injection with the turning on and off of the angiographic system, and thus limiting radiation exposure. In another aspect, the injecting of the bolus of contrast media may also automatically controlled by the controller. The controller may be same or similar to any of the controller disclosed herein.

In another aspect of the method, the injection of the embolic particle mixture is paused during the intermittent injections of the bolus of contrast media. This can result in a balanced pressure process or system in which the injection pressure is maintained substantially constant during the embolization procedure.

In still another aspect of the method, the embolic particle mixture is a pre-mix of embolic particles and delivery media disposed in the first dispenser.

In still another aspect of the method, the embolic particle mixture has a specific low concentration of embolic particles within the injected embolic particle mixture. For example, the low concentration of embolic particles to embolic particle mixture may be from 2.0%-0.05% by volume, or from 1.0-0.1% by volume. This is in the case of a total volume of embolic particle mixture injected in the range 50-500 cc (cubic cm); a volume of contrast media, which may be further diluted with saline, injected in the range of 10-50 cc; a cumulative time of injection of embolic particle mixture in the range of 5-20 minutes; and a flow rate of injection of embolic particle mixture in the range of 2.0-0.1 cc/second. Thus, the low concentration of embolic particles to embolic particle mixture may be suitably adjusted where these embolization characteristics are varied from these ranges. The size of the embolic particles may also affect the low concentration embolic particles to embolic particle mixture.

In additional aspects, the method may be performed using any of the embolization systems described herein. For instance, the embolic particle mixture may be dispensed by a first dispenser configured to dispense the embolic particle mixture into the catheter and the contrast media may be dispensed by a second dispenser configured to dispense the contrast media into the catheter. In another aspect of the method, the first dispenser may comprise a first motorized syringe configured to controllably dispense a pressurized flow of the embolic particle mixture from the first motorized syringe, and the second dispenser may comprise a second motorized syringe configured to controllably dispense a pressurized flow of the contrast media from the second motorized syringe.

In yet another aspect, the embolic particles and delivery media can be mixed upon dispensing from respective dispensers into a mixer. For example, the first dispenser may comprise a first sub-dispenser for dispensing the embolic particles into a mixer and a second sub-dispenser for dispensing the delivery media into the mixer. The mixer mixes the embolic particles and the delivery media and outputs the embolic particle mixture at the low concentration described herein. The first sub-dispenser and second sub-dispenser may comprise respective powered syringes configured to controllably dispense a pressurized flow of fluid.

In still another aspect, the delivery media (e.g., saline) may be injected at a controlled rate into the mixer or manifold from the second sub-dispenser, and the embolic particles are then injected from the first sub-dispenser into the mixer at a controlled rate to create and dispense the embolic particle mixture. Then, to switch to dispensing the contrast media, the second dispenser injects the contrast media into the mixer or manifold. The second sub-dispenser may simultaneously inject the delivery media (e.g., saline) into the mixer or manifold while the second dispenser injects the high concentration contrast media into the mixer or manifold, thereby creating and dispensing a reduced concentration contrast media mixture. The second sub-dispenser can inject delivery media continuously, and at a varying or constant rate, while switching from dispensing embolic particles by the first sub-dispenser in a first step, to dispensing contrast media from the second dispenser. The controller can control the rates of dispensing of embolic particles plus delivery media, or contrast media plus delivery media, to create the appropriate dilution/concentration of the respective mixtures. The controller may be programmable to set the desired rates of dispensing and dilution/concentration of the respective mixtures.

In additional aspects of the method, the first dispenser and second dispenser may be electronically controlled to controllably dispense the respective fluids. In yet another aspect, of the method, the first dispenser and second dispenser may be operably coupled to a controller which automatically controls the operation of the first dispenser to controllably dispense the embolic particle mixture from the first dispenser and automatically controls the operation of the second dispenser to controllably dispense the contrast media.

In another aspect, the method may further include selectably programming the controller to control a characteristic of the dispensing of the embolic particle mixture by the first dispenser and the dispensing of the contrast media by the second dispenser. As some examples, the characteristic of dispensing by the first dispenser and second dispenser may be (a) a flow rate of dispensing, (2) a pressure of dispensing, (3) a particle concentration in the embolic particle mixture, (4) frequency of dispensing contrast media, (5) a bolus volume or time for the dispensing of contrast media etc.

In additional aspects, the method may further include the controller receiving a first sensor signal from a first sensor related to an attribute of the respective dispensing by the first dispenser and second dispenser. The controller may use a feedback control process to control the dispensing by the first dispenser and second dispenser based on the first sensor signal. In other aspects, the first sensor may be a flow rate sensor, a pressure sensor, a force sensor, actuator torque sensor, and/or a motor current sensor. In other aspects of the method, the attribute of dispensing controlled by the controller may be an injection flow rate or an injection pressure of the dispensing by the first dispenser and second dispenser into the catheter. The controller controls the first dispenser and second dispenser using the feedback control process to achieve a prescribed characteristic of dispensing into the catheter. The prescribed characteristic of dispensing into the catheter may include any one or more of flowrate, pressure and concentration of dispensed embolic particle mixture or contrast media.

In another aspect, the embolization system may also include an on/off switch, such as a button, touch screen input, or the like, to start and stop the injection of embolic particle mixture. The starting and stopping of injecting embolic particle mixture may be manually controlled, or automatically controlled by the controller.

In still another aspect, the embolization system may be configured to perform a priming process to remove any air within the system. For example, the system may be configured to automatically inject delivery media (e.g., saline), into the catheter(s) and other components to purge air from the dispensing components of the embolization system.

In yet another aspect, the embolization system may also be configured to emit a contrast media signal indicating that the bolus of contrast media is about to be injected. The signal may be used to inform the user or an angiographic system controller that the bolus of contrast media is about to be injected so that the angiography system can be activated manually or automatically to visualize the embolization procedure. The contrast media signal may be an audible sound, a lighted indicator, or an electronic trigger. This allows the angiography system to be turned on only when needed, temporally proximate to the injection of contrast media, which limits the radiation exposure of the patient and user. The controller of the embolization system may be configured to communicate with the angiographic system in order to transmit the contrast media signal to the angiographic system which activates the angiographic system.

Accordingly, improved embolization systems and methods are disclosed herein which can dispense a low concentration of embolic particle mixture. The systems and methods use a low concentration of embolic particles in the embolic particle mixture thereby reducing the possibility of clogging (i.e., embolization of non-targeted vessels) and reflux. Furthermore, the embolization systems and method have the capability of injecting a bolus of contrast media separately from the injection of embolic particle mixture. In this way, a puff or bolus of contrast media can be injected without having to inject a relatively large burst of embolic particle mixture which is prone to clogging and reflux. Also, the use of electronical controls and automation, the systems and method may be comfortably used by a practitioner for an extended period of time to inject a very low concentration of embolic particle mixture for a longer period of time than the previous manually controlled systems without causing fatigue and also reducing the risk of human error during an embolization procedure inherent in such manual systems, such as those described above.

### BRIEF DESCRIPTION OF THE DRAWINGS

The drawings illustrate the design and utility of various aspects of the disclosure, in which similar elements are referred to by common reference numerals. These drawings are not necessarily drawn to scale. In order to better appreciate how the above-recited and other advantages and objects are obtained, a more particular description of the disclosure will be rendered, which is illustrated in the accompanying drawings. These drawings depict only exemplary aspects of the disclosure for purposes of illustration and facilitating the below detailed description, and are not therefore to be considered limiting of its scope.

FIG. 1 is a schematic side view of an embolization system according to one example disclosed herein.

FIG. 2 is an illustration depicting a balanced pressure system dispensing scheme for the embolization system of FIG. 1.

FIG. 3 is an illustration depicting a balanced flow rate dispensing scheme for the embolization system of FIG. 1.

FIG. 4 is a schematic side view of an embolization system according to another example disclosed herein.

FIG. 5 is a schematic side view of an embolization system according to still another example disclosed herein.

FIG. 6 is an illustration depicting a balanced pressure system dispensing scheme for the embolization system of FIG. 5.

FIG. 7 is an illustration depicting a balanced flow rate dispensing scheme for the embolization system of FIG. 5.

FIG. 8 is a schematic side view of an outlet of an embolic particle mixture flow showing how embolic particles can aggregate and clog.

FIG. 9 is a schematic side view of a flow rectifier for use with the embolization systems disclosed herein.

FIG. 10 is a schematic side view of distal end of an embolization system attached to a hub and catheter assembly, according to another example disclosed herein.

### DETAILED DESCRIPTION OF THE DISCLOSURE

This specification describes exemplary embodiments, aspects and applications of the disclosure. The disclosure, however, is not limited to these exemplary embodiments, aspects and applications or to the manner in which the exemplary embodiments, aspects and applications operate or are described herein. Further, the figures may show simplified or partial views, and the dimensions of elements in the figures may be exaggerated or otherwise not in proportion. Moreover, elements of similar structures or functions are represented by like reference numerals throughout the figures. In addition, an illustrated aspect need not have all the features or advantages shown. A feature or an advantage described in conjunction with a particular aspect is not necessarily limited to that aspect, and can be practiced in any other aspect even if not so illustrated.

For the following defined terms, these definitions shall be applied, unless a different definition is given in the claims or elsewhere in this specification.

Where reference is made to a list of elements (e.g., elements a, b, c), such reference is intended to include any one of the listed elements by itself, any combination of less than all of the listed elements, and/or a combination of all of the listed elements.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. The term “ones” means more than one.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure. The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

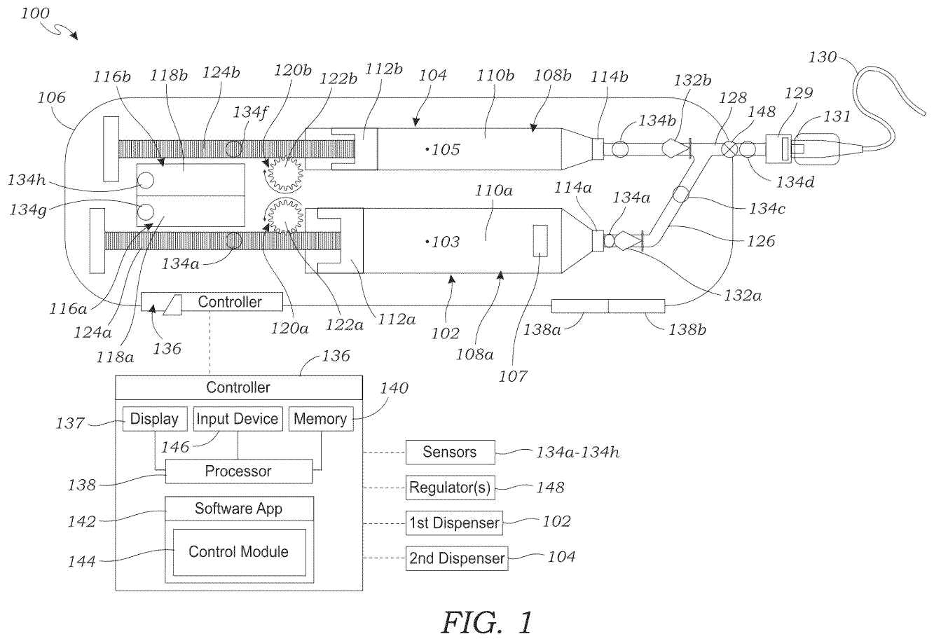

Referring to FIG. 1, one example of an embolization system 100 for performing medical embolization procedures to embolize a target region of tissue in a human body is illustrated. The embolization system 100 includes a first dispenser 102 and a second dispenser 104, housed within an enclosure 106. The first dispenser 102 is for containing and dispensing a fluid comprising an embolic particle mixture 103 including a mixture of embolic particles and a delivery media. The second dispenser 104 is for dispensing a fluid comprising contrast media 105 for visualizing an embolization procedure using the embolization system 100.

In the illustrated embodiment of FIG. 1, each of the first dispenser 102 and second dispenser 104 comprise a motorized syringe 108a, 108b, respectively. Each motorized syringe 108a, 108b includes a barrel 110a, 110b (also called a chamber 110a, 110b) for containing the respective fluid to be dispensed and a plunger 112a, 112b disposed in the barrel 110a, 110b for pushing the fluid out of the barrel 110a, 110b. Each syringe 108a, 108b also has a syringe outlet port 114a, 114b at the distal end of the barrel 110a, 110b.

Each motorized syringe 108a, 108b also includes an actuator 116a, 116b for actuating the plunger 112a, 112b. In the illustrated example, the actuators 116a, 116b comprise an electric motor 118a, 118b and a coupling 120a, 120b which couples the electric motor 118a, 118b to the plunger 112a, 112b. The coupling 120a, 120b may be one or more gears 122a, 122b (e.g., a pinion or worm gear), or other coupling devices for converting the rotational motion of the electric motor 118a, 118b to linear motion of the plunger 112a, 112b. The plunger 112a, 112b may have a rack type gear 124a, 124b which mates with gear 122a, 122b to convert the rotational motion of the gear 122a, 122b to linear motion of the plunger 112a, 112b.

The outlet port 114a is connected to, and in fluid communication with, a manifold 126, and the outlet port 114b is also connected to, and in fluid communication with, the manifold 126, such that the first dispenser 102 and the second dispenser 104 dispense the respective fluids into the manifold 126. The manifold 126 has an outlet 128 which ejects the fluid from the first dispenser 102 and second dispenser 104 out of the manifold 126 and into a catheter 130. The embolization system 100 has a connector 129 which is configured to be connected to the proximal hub 131 of the catheter 130.

In an optional feature, the first dispenser 102 may also include a mixer 107 configured to mix the embolic particle mixture 103 within the first dispenser 102. For example, the embolic particles being used may have a tendency to settle over time. The mixer 107 assists in maintaining a uniform concentration of particles within the embolic particle mixture 103 within the first dispenser 102 to be injected. The mixer 107 may be any suitable mixer, such as a stir bar, a recirculation or pulsed pump, a vibratory, tilting, a device to rotate or shake the first dispenser 102, etc.

The embolization system 100 has a control module 136 (also referred to as the “controller 136”) for monitoring and controlling the operation of embolization system 100. The control module 136 may include a pair of manual on/off switches 138a, 138b for turning on and off the first dispenser 102 and second dispenser 104, respectively. This allows a user to manually turn on and off the first dispenser 102 to controllably dispense the embolic particle mixture 103 into the catheter 130, and to manually turn on and off the second dispenser 104 to controllably dispense the contrast media 105. The manual operation may be useful in priming the system 100 by purging any trapped air from the system 100. The on/off switches 138a, 138b may be adjustable to vary the rate of actuation of the motorized syringes 108a, 108b to control the dispensing flow rate of the first dispenser 102 and second dispenser 104. For example, the on/off switches 138a, 138b may be a variable speed button or slide switch which varies the speed of the motors 116a, 116b, respectively, which varies the rate at which the plungers 112a, 112b are advanced thereby varying the dispensing flow rate. The control module 136 may also include a flow rate indicator which shows the dispensing flow rate. In the case of syringes 108a, 108b as the dispensers 102, 104, the flow rate is directly proportional to the speed of the plunger 112a, 112b, and can be directly calculated based on the dimensions of the barrel 110a, 110b of the syringe 108, 108b.

The embolization system 100 may also include one-way valves 132a, 132b between the syringe outlet port 114a, 114b and manifold 126 to prevent backflow of fluid from one of the dispensers 102, 104 to the other dispenser 102, 104.

In order to monitor and control the dispensing by embolization system 100, including automated dispensing as described herein, the system 100 includes sensors 134 for sensing an attribute of the fluid at the location of the respective sensor 134 and to output a sensor signal related to the attribute. In the example of FIG. 1, the embolization system 100 includes a first sensor 134a, a second sensor 134b, a third sensor 134c, and a fourth sensor 134d. The sensors 134a-134d may be any suitable sensors such as pressure sensors, flow rate sensors, light absorbance sensor or the like. In one example, the sensors 134a, 134b and 134c are each pressure sensors for detecting the fluid pressure at the outlet of the first dispenser 102, the fluid pressure at the outlet of the second dispenser 104 and the fluid pressure in the manifold 126, respectively. The fourth sensor 134d is a differential pressure flow rate sensor for detecting the flow rate at the outlet of the manifold 126.

In order to control the dispensing by the system 100, each of the first and second dispensers 102, 104 and the sensors 134a-134d are operably coupled to the controller 136. The controller 136 is an electronic controller having a microprocessor or electronic control logic (e.g., logic circuitry) 138, memory 140, and a software application 142. The controller 136g may also be configured to receive the sensor output signals from each of the sensors 134a-134d and to use the sensor signals to monitor and control the operation of the embolization system 100. As some examples, the controller 136 may use the output signals to determine the flow rate and/or fluid pressure of the embolic particle mixture 103 being dispensed by the first dispenser 102, the flow rate and/or fluid pressure of the contrast media being dispensed by the second dispenser 104, the flow rate and/or fluid pressure of the fluid being dispensed into the catheter 130. The software application 142 includes a control module 144 for controlling the operation of the embolization system 100 based on any one or more of the sensor signals from the sensor 134a-134d. The controller 136 utilizes the control module 144 to control the operation first dispenser 102 and second dispenser 104, including controlling the flow rate of dispensing, the pressure of dispensing, the timing of on/off cycling of the dispensing, and/or the volume of fluid dispensed during the on/off cycling of the dispensing. The control module 144 may include a feedback control algorithm which controls the dispensing of the first dispenser 102 and second dispenser 104 based on any one or more of the sensor signals from the sensors 134a-134d.

The controller 136 is selectably programmable by the user to set one or more operating characteristics of the operation of the embolization system 100 during use for embolizing a target region of tissue. The controller 136 includes an input device 146 which allows the user to select various operating parameters of the embolization system 100. The input device 146 may be any suitable input device such as a keyboard, touchscreen, button pad, touchpad, etc. As some examples, the user may set the dispensing flow rate of the first dispenser 102 and/or second dispenser 104, the dispensing pressure of the first dispenser 102 and/or second dispenser 104, the timing duration and/or cycling rate of on/off cycling of the first dispenser 102 and/or second dispenser 104, the cycle time between dispensing a bolus of contrast media 105 by the second dispenser 104 and a successive bolus of contrast media 105 and/or dispensing embolic particle mixture 103, the size (e.g., volume or time of dispensing) of the bolus of contrast media 105 by the second dispenser 104, the amount of embolic particle mixture 103 to be dispensed in total (by volume, weight or other suitable measure), the total dispensing time for an embolization procedure, etc. The software application 142 may include a number of pre-programmed dispensing programs having various pre-set dispensing parameters from which the user can select. The pre-programmed dispensing programs can have user selectable characteristics such as flow rates, pressures, total amount of embolic particle mixture, frequency of injection of contrast media 105, etc.

The embolization system 100 may also have other sensors 134e-134h for detecting operating attributes of the motorized syringes 108a, 108b and outputting respective control signals related to the operating attributes. These other sensors 134e-134h are also operably coupled to the controller 136 and the controller 136 is configured to receive the sensor output signals from each of the sensors 134e-134h and to use the sensor signals to monitor and control the operation of the embolization system 100. For example, sensors 134e and 134f may be torque sensors which measure the torque on the plungers 112a, 112b, respectively. The torque on the plungers 112a, 112b can be used by the controller 136 to indirectly determine the outlet pressure of the first dispenser 102 and second dispenser 104. Similarly, the sensors 134g and 134h may be motor current sensors which measure the current to the motors 118a, 118b, respectively. The motor current may also be used to indirectly determine operating attributes of the motorized syringes 108a, 108b, such as the flow rate of dispensing by the first dispenser 102 and second dispenser 104, respectively. The sensors 134g, 134h may be motion sensors configured to detect motion of the plungers 112a, 112b (linear motion) or rotations of the motors 118a, 118b (rotational sensor) which may be used to determining the flow rate of dispensing by the first dispenser 102 and second dispenser 104, respectively. The control module 144 may utilize the output signals from the sensors 134e-134h to monitor and control the dispensing of the first dispenser 102 and second dispenser 104, including in the feedback control algorithm.

The embolization system 100 may include one or more pressure relief valves and/or regulators 148 to regulate the pressure of the flows of fluid within or out of the system 100. In the example of FIG. 1, a relief valve or regulator 148 may be installed in the outlet of the manifold 126 to regulate the pressure of the fluid being dispensed out of the embolization system 100. Relief valves or regulators 148 may be installed in any one or more of the outlet of the first dispenser 102, the outlet of the second dispenser 104, the outlet of the manifold 126, and within the manifold 126 for regulating the flows and pressures of the dispensed fluids within or out of the system, adjusting the concentrations of the fluids (e.g., the concentration of a mixture of the embolic particle solution and the contrast media), and/or facilitating the mixing of the constituent fluids. In the case of relief valve(s) 148, the relief valve(s) 148 also function as passive overpressure prevention device(s).

The embolization system 100 may also include a pressure or force limiter between the electric motor 118a, 118b and the coupling 120a, 120b to the plunger 112a, 112b, to prevent over-pressurization of the fluids being dispensed, such as a torque limiter, slip clutch, or the like.

The use and operation of the embolization system 100 in performing an embolization procedure to embolize a target region of tissue will now be described. The catheter 130 is inserted through an incision in a patient's body and advanced to a target position proximate the target region of tissue to be embolized. The catheter 130 may be positioned within the patient's body before or after connecting the catheter 130 to the embolization system 100, although it may be more convenient to position the catheter 130 before connecting so that the embolization system 100 does not interfere with the manipulating the catheter 130. The embolization system 100 is then connected to the catheter 130. The system 100 can also be connected via a secondary fluid connection to the catheter 130 such as a rotating hemostatic valve (RHV) or a fluid management stopcock or manifold.

The user may then use the embolization system 100 in a manual or automatic mode to embolize the target region of tissue. The first dispenser 102 is filled with embolic particle mixture 103. The embolic particle mixture 103 may have a low concentration of embolic particles within the embolic particle mixture 103 in a range from 2.0%-0.05% by volume, or in a range from 1.0-0.1% by volume. There are a number of factors to consider in setting a target concentration of embolic particles to embolic particle mixture 103, such as the total volume of embolic particle mixture 103 to be used in an embolization procedure, the flow rate of injection and the size and amount of embolic particles desired for the particular clinical scenario or particular target region or particular volume of tissue to be embolized. The ranges of concentration of embolic particles within the embolic particle mixture 103 disclosed herein are estimated for a total volume of embolic particle mixture 103 to be injected in the range 100-500 cc (cubic cm); a cumulative time of injection of embolic particle mixture in the range of 5-20 minutes; and a flow rate of injection of embolic particle mixture in the range of 2.0-0.1 cc/second. Thus, the low concentration of embolic particles to embolic particle mixture may be suitably adjusted where these embolization characteristics are varied from these ranges. The total volume of media contrast diluted with saline may be injected in the range of 10-50 cc.

The second dispenser 104 is filled with a suitable contrast media 105 for the visualization system used to monitor the procedure. In a manual mode, the user actuates the first dispenser 102 and/or second dispenser 104 as needed to dispense the desired amounts of embolic particle mixture 103 and contrast media 105, respectively. The controller 136 may be configured to provide dispensing information to the user, such as the pressure, flow rate, volume of dispensed fluid, etc. The dispensing information may be displayed on a display 137 operably coupled to the controller 136 (e.g., the display 137 may be part of the controller 136), or provided to the user in some other suitable manner such as by communicating the dispensing information to an external device (e.g., a computing device such as a laptop, smartphone, etc.) via a communication link (e.g., a wired or wireless link such as WiFi, Bluetooth, etc.). The user may use the dispensing information to monitor and control the embolization procedure using the embolization system 100.

In one manual mode of using the embolization system 100, the user operates manual on/off switch 138a to actuate the first motorized syringe 108a of the first dispenser 102 to dispense embolic particle mixture 103 into the catheter 130 which is delivered by the catheter 130 to the target region. The user may control the flow rate of dispensing the embolic particle mixture using the switch 138a or by programming the controller 136 accordingly. The user intermittently operates the manual on/off switch 138b to actuate the second motorized syringe 108b of the second dispenser 102 to dispense a bolus of contrast media 105 into the catheter 130 which is delivered by the catheter 130 to the target region. Upon dispensing a bolus of contrast media 105 and ejection of the bolus of contrast media 105 from the catheter at the target position, the user can track the contrast media 105 as it is injected into the target region to observe and monitor the progress of embolization of the target region of tissue using a visualization system, such as a fluoroscope or other suitable X-ray system adapted to detect the contrast media and anatomy within the patient and generate real-time images displayed on a monitor. The user can temporarily pause the dispensing of the embolic particle mixture 103 while dispensing the bolus of contrast media 105, and then re-start dispensing of the embolic particle mixture 105 to push the bolus of contrast media 105 through the catheter 130 and into target region of tissue. Alternatively, the user can continuously dispense the embolic particle mixture 103 while also dispensing the bolus of contrast media 105.

To use the embolization system 100 in an automatic mode, the user uses the input device 146 to select the desired embolization system 100 operating parameters, such as dispensing flow rate of the first dispenser 102 and/or second dispenser 104, the dispensing pressure of the first dispenser 102 and/or second dispenser 104, the on/off cycling of the first dispenser 102 and/or second dispenser 104, the cycle time between dispensing a bolus of contrast media 105 by the second dispenser 104, the size (e.g., volume or time of dispensing) of the bolus of contrast media 105 by the second dispenser 104, the amount of embolic particle mixture 105 to be dispensed in total (by volume, weight or other suitable measure), the total dispensing time for an embolization procedure, etc., or a pre-programmed dispensing program and if desired, setting any one or more of the user selectable characteristics of the dispensing program such as flow rates, pressures, total amount of embolic particle mixture, frequency of injection of contrast media 105, etc.

The user then starts an automated embolization procedure on the embolization system 100 by inputting a “start” command using the input device 146. The controller 136 automatically operates the embolization system 100 to perform the selected automated embolization dispensing program using the first dispenser 102, second dispenser 104, sensors 134, valves and regulators 148 as controlled by the controller 136. In one generalized automated embolization procedure, the embolization system 100 actuates the first dispenser 102 to dispense the embolic particle mixture 103 into the catheter 130 to inject the embolic particle mixture 103 into the target region at a flow rate, pressure and/or volume controlled by the controller 136, which may be accomplished using the feedback control process based on the sensor signals from any one or more of the sensors 134a-134e. The embolization system 100 intermittently actuates the second dispenser 104 to dispense a bolus of contrast media 105 into the catheter 130 to inject the contrast media 105 into the target region, which also which may be accomplished using the feedback control process based on the sensor signals from any one or more of the sensors 134a-134e.

The embolization system 100 may also be configured to emit a contrast media signal indicating that the bolus of contrast media 105 is about to be injected. The signal may be used to inform the user or an angiographic system controller that the bolus of contrast media 105 is about to be injected so that the angiography system can be activated manually or automatically to visualize the embolization procedure. The contrast media signal may be an audible sound, a lighted indicator, or an electronic trigger. This allows the angiography system to be turned on only when needed, temporally proximate to the injection of contrast media, which limits the radiation exposure of the patient and user. The controller 136 of the embolization system 100 may be configured to communicate with the angiographic system in order to transmit the contrast media signal to the angiographic system which activates the angiographic system. Upon injecting the bolus of contrast media 105 from the catheter 130 at the target position, the user can track the contrast media 105 as it is injected into the target region and propagates through the target region of tissue to observe and monitor the progress of embolization of the target region of tissue using a visualization system, such as a fluoroscope or other suitable X-ray system adapted to detect the contrast media and anatomy within the patient and generate real-time images displayed on a monitor.

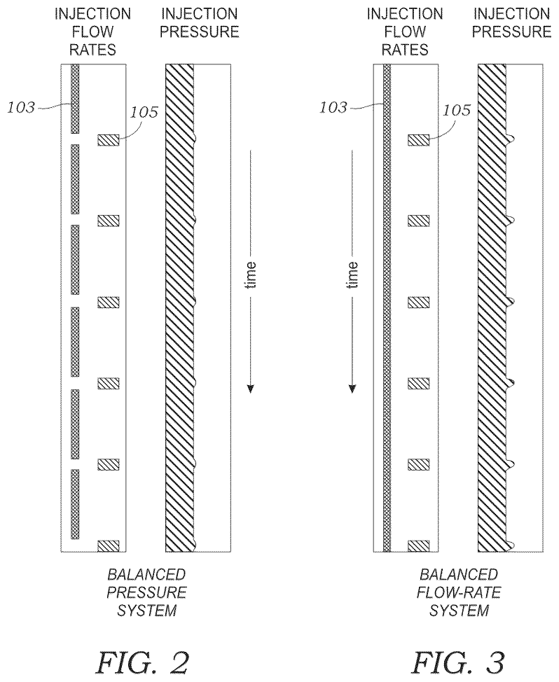

Turning to FIGS. 2 and 3, two different control schemes for dispensing the embolic particle mixture and the contrast media are depicted. FIG. 2 shows a chart depicting a balanced pressure scheme in which the dispensing/injection pressure is controlled and the flow rate is whatever flow rate stems from controlled dispensing/injection pressure. The balanced pressure scheme can be used to ensure that a safe pressure is always maintained for the fluid being injected into a particular anatomy of the target region. As shown in FIG. 2, in a balanced pressure scheme, the dispensing of the embolic particle mixture 103 by the first dispenser 102 is paused during dispensing of the bolus of contrast media 105 by the second dispenser 104. This may maintain a substantially constant injection pressure at the output of the manifold 126, with the potential for only a small increase in pressure during injection of the bolus of contrast media 105. The embolization system 100 may be configured to operate using the balance pressure scheme using the relief valve and/or regulator 148 and/or by configuring the controller 136 to control the dispensing to maintain a set dispensing/injection pressure. For example, relief valve and/or regulator 148 can be set to control the dispensing pressure and/or the controller 136 can control the actuation force of the motorized syringes 108a, 108b, such as by controlling the current on the motors 134h, 134g to obtain the required dispensing pressure by the first dispenser 102 and second dispenser 104.

In contrast, FIG. 3 shows a chart depicting a balanced flow rate scheme in which the dispensing/injection flow rate is controlled and the pressure is whatever pressure stems from the controlled dispensing/injection flow rate. This scheme may be preferred in order to ensure a desired flow rate of embolic particle mixture 103 (or number of embolic particles dispensing rate, which is proportional to the flow rate of embolic particle mixture 103, assuming a relatively constant concentration) during an embolization procedure using the embolization system 100. As shown in FIG. 3, in a balanced flow rate scheme, the dispensing of the embolic particle mixture 103 by the first dispenser 102 may be continuous during dispensing of the bolus of contrast media 105 by the second dispenser 104 in order to maintain the set flow rate of embolic particle mixture. As also shown in FIG. 3, this results in an increase in the injection pressure at the output of the manifold 126 during injection of the bolus of contrast media 105. The embolization system 100 can be configured to operate in the balanced flow rate scheme by configuring the controller 136 to control the dispensing flow rate of the first dispenser 102 and second dispenser 104, such as by controlling the speed at which the respective motorized syringes 108a, 108b are actuated, i.e., the speed at which the plungers 112a, 112b are advanced, which may be proportional to the speed of the respective motors 134g, 134h.

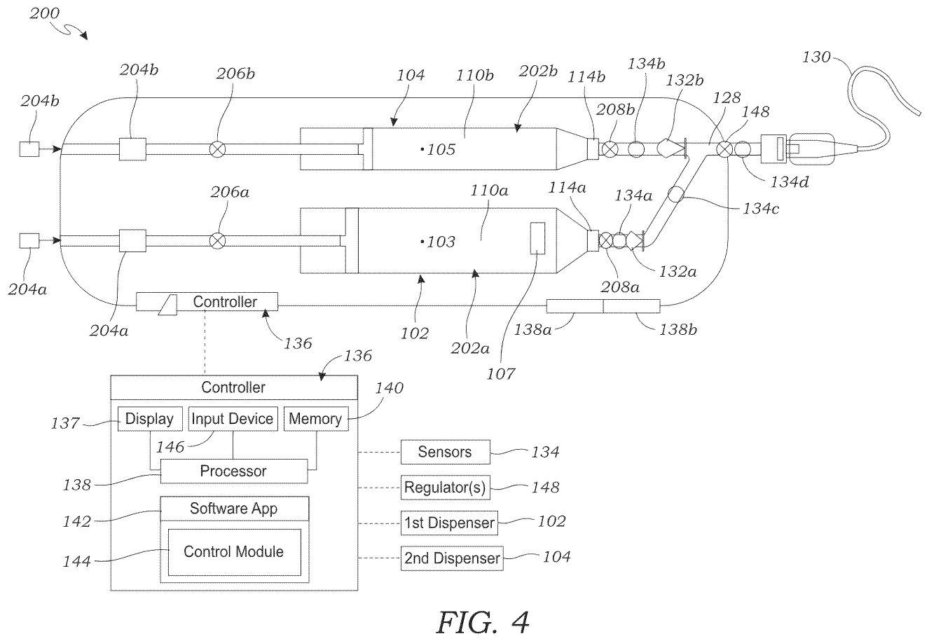

Turning now to FIG. 4, another example of an embolization system 200 will be disclosed. The embolization system 200 is the same as the embolization system 100, except that the first dispenser 102 and second dispenser 104 are not motorized syringes 108a, 108b, but are instead a first pressurized dispenser 202a and a second pressurized dispenser 202b. Each of the first and second pressurized dispensers 202a, 202b comprise a pressure chamber 110a, 110b and a pressure source 204a, 204b. The pressure source 204a 204b may be a gas (e.g., in house compressed air) source, a fluid pump or a pressure canister/vessel/chamber/bag for pressurizing the pressure chamber 110a, 110b. As shown in FIG. 4, the pressure source 204a 204b may be disposed on the embolization system 200 or it may be external to the embolization system 200 in which case the embolization system 100 has fittings to connect the pressure source 204a, 204b to the embolization system a 100 to the external pressure source 204a, 204b. A pressure relief valve 206a, 206b and/or regulator may be used to control the pressure provided by the pressure source 206a, 206b to pressurize the pressure chamber 110a, 110b.

The first and second pressurized dispensers 202a, 202b also include a first dispensing valve 208a and a second dispensing valve 208b, respectively, at the respective outlets 114a, 114b. of the dispensers 202a, 202b. The first and second dispensing valves 208a, 208b. The first and second dispensing valves 208a, 208b are operably coupled to the controller 136 to enable the controller to open and close the dispensing valves 208a, 208b to dispense the respective fluids from the pressurized dispensers 202a, 202b. The first and second pressurized dispensers 202a, 202b operate to dispense the embolic particle mixture 103 and the contrast media 105, respectively, by pressurizing the chamber 110a, 110b, and then opening the respective dispensing valve 208a, 208b to dispense a pressurized flow of embolic particle mixture 103 and contrast media 105 and closing the respective dispensing valve 208a, 208b to stop the dispensing.

The monitoring and control of the embolization system 200 is the same as that described for the embolization 100, except that the dispensing is controlled by controlling the first and second pressurized dispensers 202a, 202b, instead of the motorized syringes 108a, 108b. Similarly, the use and operation of the embolization system 200 in performing an embolization procedure to embolize a target region of tissue is the same as for the embolization system 100, except that the dispensing is performed by controllably operating the first and second pressurized dispensers 202a, 202b, instead of the motorized syringes 108a, 108b.

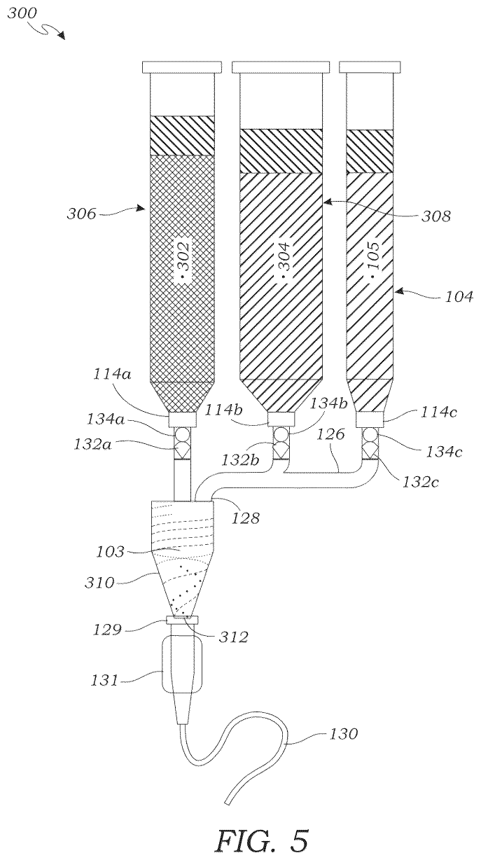

Referring now to FIG. 5, another example of an embolization system 300 will be described. The embolization system 300 is very similar to the embolization system 100, except that the embolization system 300 is configured to separately dispense the embolic particles 302 (in a carrier fluid at a high concentration of embolic particles 302) and a delivery media 304 and mix the embolic particles and delivery media to prepare and dispense the embolic particle mixture 103. This is analogous to the embolization system 100 described herein in which the first dispenser 102 comprises a first sub-dispenser for dispensing embolic particles and a second sub-dispenser for dispensing the delivery media, and mixing the embolic particles and delivery media to produce the embolic particle mixture dispensed by the first dispenser 102. This allows the embolization system 300 to vary the concentration of the embolic particle mixture and also allows the user to selectably control the concentration of the embolic particle mixture. FIG. 5 is a partial view of the embolization system 300 and does not show all of the details of the actuation mechanisms of the first sub-dispenser 306, second sub-dispenser 308, and the second dispenser 104, nor does it show the manual on/off switches 138a, 138b or the controller 136. It is understood that the actuation mechanisms for the first sub-dispenser 306, second sub-dispenser 308, and the second dispenser 104 may be the same or similar to the actuation mechanisms of the embolization system 100 (i.e., motorized syringes 108a, 108b) and/or the embolization system 200 (i.e., pressurized dispensers 202a, 202b), as described herein. It is also understood that the embolization system 300 includes the same or similar manual on/off switches 138a, 138b and controller 136 as the embolization systems 100, 200, and such elements function as described herein.

As shown in FIG. 5, the embolization system 300 includes a first sub-dispenser 306 for dispensing embolic particles 306. The combination of the first sub-dispenser 306 and second sub-dispenser 308 may be considered the first dispenser 102 of the embolization system 300. The embolization system 300 also includes additional sensors 134 for the first and second sub-dispensers 306 and 308 for sensing an attribute of the fluid at the location of the respective sensor 134 and to output a sensor signal related to the attribute. The additional sensors 134 are also operably coupled to the controller 136. The manual on/off switches 138 may include separate switches for the 138 for actuating the first sub-dispenser 306 and actuating the second sub-dispenser 308 such that a user can manually vary the concentration of embolic particles 304 in the embolic particle mixture 103 by using the switches. Alternatively, one manual switch 138a may actuate both the first sub-dispenser 306 and second sub-dispenser 308, and the flow rates of the respective first sub-dispenser 306 and second sub-dispenser 308 may be controlled by the controller 136 to achieve a selected concentration. For instance, the user may select a concentration (which may be constant or varying, as described herein) using the input device 146, and the controller 136 can control the respective dispensing flow rates of the first sub-dispenser 306 and second sub-dispenser 308 to achieve the selected concentration.

The embolization system 300 also includes a mixer 310 connected to, and in fluid communication with, the outlet 128 of the manifold 126. The mixer 310 may be any suitable mixer, configured to mix the embolic particles 302 and the delivery media 304 and output the embolic particle mixture 103 through an outlet 312 of the mixer 310. The mixer 310 may be a passive mixer such as a flow induced mixer, static mixer, or vortex mixer, or an active mixer such as a mixer having a moving mixing element, or a mixer having a non-moving mixing element of sound wave transducer, or mechanical wave transmitter.

The monitoring and control of the embolization system 300 is the same as that described for the embolization 100, except for adding the functionality of separately dispensing the embolic particles 306 by the first sub-dispenser 306 and the delivery media 304 by the second sub-dispenser 308, and mixing the dispensed embolic particles 306 and the delivery media 304 to produce the embolic particle mixture 103 dispensed out of the mixer and into the catheter 130. Thus, the controller 136 and software application 142 are also configured to vary the concentration of the embolic particle mixture and allow the user to selectably control the concentration of the embolic particle mixture. As some examples, controller 136 and software application 142 are configured to allow the user to use the input device 146 to select a concentration of embolic particle mixture 103 which may be a constant concentration or a varying concentration selected by the user. The flow rate of the mixed embolic particle mixture 103 may also be selected by the user or set in the pre-programmed dispensing programs. For instance, the controller 136 and software application 142 may include pre-set programs which vary the concentration through an embolization procedure, such as progressively ramping the concentration up or down during portions of an embolization procedure, and/or using a constant concentration during portions of an embolization procedure. The variation of the concentration may be within a user selectable range of concentrations defining a minimum and maximum concentration, or defining a minimum and maximum flow rate of a number of embolic particles being dispensed (i.e., number of embolic particles per unit of time). The controller 136 can then determine the concentration of the embolic particle mixture 103 and the flow rate of the embolic particle mixture 103 being dispensed, to achieve the programmed flow rate of number of embolic particles.

Similarly, the use and operation of the embolization system 300 in performing an embolization procedure to embolize a target region of tissue is substantially the same as for the embolization system 100, except that embolic particles 302 and delivery media 304 are dispensed separately by the first sub-dispenser 306 and second sub-dispenser 308, and are mixed to produce the embolic particle mixture 103 dispensed out of the mixer 310 and into the catheter 130, and the embolization system enables the concentration of embolic particles 302 in the embolic particle mixture 103 to be varied during the procedure. The catheter 130 is inserted and positioned at a target position proximate a target region of tissue to be embolized, as described herein for the embolization system 100. The first sub-dispenser 306 is filled with embolic particles 302, the second sub-dispenser 308 is filled with a delivery media 304, such as saline, water and/or a medicament, and the second dispenser 104 is filled with a suitable contrast media 105 for the visualization system used to monitor the procedure. The user may then use the embolization system 300 in a manual or automatic mode to embolize the target region of tissue.

In a manual mode, the user actuates the first sub-dispenser 306 and second sub-dispenser 308 as needed to dispense selected relative amounts of embolic particles 302 and delivery media 304 to dispense a selected concentration of embolic particle mixture 103. For example, the relative amounts of embolic particles 302 and delivery media 304 may be dispensed and mixed to produce an embolic particle mixture 103 having a low concentration of embolic particles to embolic particle mixture 103 in a range from 2.0%-0.05% by volume, or in a range from 1.0-0.1% by volume. The user intermittently actuates the second dispenser 104 to intermittently dispense a bolus of contrast media 105. The controller 136 may be configured to provide dispensing information to the user, such as the pressure, flow rate, volume of dispensed fluid, etc. The dispensing information may be displayed on a display 137 operably coupled to the controller 136 (e.g., the display 137 may be part of the controller 136), or provided to the user in some other suitable manner such as by communicating the dispensing information to an external device (e.g., a computing device such as a laptop, smartphone, etc.) via a communication link (e.g., a wired or wireless link such as WiFi, Bluetooth, etc.). The user may use the dispensing information to monitor and control the embolization procedure using the embolization system 100.

In one example of a manual mode of using the embolization system 300, the user operates manual on/off switch 138 a to actuate the first sub-dispenser 306 to dispense embolic particles 302 and the second sub-dispenser 308 to dispense delivery media 304 which are mixed in the mixer 310 to produce and dispense embolic particle mixture 103 into the catheter 130 which is delivered by the catheter 130 to the target region. The user may select a concentration of embolic particle mixture 103 using the controller 136, and the controller 136 then controls the relative amounts of embolic particles 302 dispensed by the first sub-dispenser 306 and the delivery media 304 dispensed by the second sub-dispenser 308 to produce the selected concentration. The controller 136 produces the selected concentration by controlling the relative flow rates embolic particles 302 dispensed by the first sub-dispenser 306 and the delivery media 304 dispensed by the second sub-dispenser 308. The user operates the manual on/off switch 138 a to simultaneously actuate the first sub-dispenser 306 to dispense the embolic particles 302 and the second sub-dispenser 308 to dispense the delivery media 304. The embolic particles 302 and delivery media 304 flow through the manifold 126 and are mixed in the mixer 310 to produce the selected concentration of embolic particle mixture 103. The embolic particle mixture 310 is injected into the catheter 130 and flows through the catheter 130 and out of the distal end of catheter 130 into the target region of tissue. Alternatively, the user may manually and separately operate both the first sub-dispenser 306 and second sub-dispenser 304 to manually control the relative amounts of embolic particles 302 and delivery media 304, to produce a manually controlled concentration of embolic particle mixture 103. In this case, the manual on/off switch 138a may include separate switches for the first sub-dispenser 306 and second sub-dispenser 308. The user may control the flow rate of dispensing the embolic particle mixture 103 using the switch 138a or by programming the controller 136, accordingly.

The user intermittently operates the manual on/off switch 138b to actuate the second the second dispenser 104 to dispense a bolus of contrast media 105 into the catheter 130 which is delivered by the catheter 130 to the target region. Upon dispensing a bolus of contrast media 105 and ejection of the bolus of contrast media 105 from the catheter at the target position, the user can track the contrast media 105 as it is injected into the target region to observe and monitor the progress of embolization of the target region of tissue using a visualization system, such as a fluoroscope or other suitable X-ray system adapted to detect the contrast media and anatomy within the patient and generate real-time images displayed on a monitor. The user can temporarily pause the dispensing of the embolic particle mixture 103 while dispensing the bolus of contrast media 105, and then re-start dispensing of the embolic particle mixture 103 (or just the delivery media 304) to push the bolus of contrast media 105 through the catheter 130 and into target region of tissue. Alternatively, the user can continuously dispense the embolic particle mixture 103 while also dispensing the bolus of contrast media 105.

In another aspect, the sub-dispenser mode of operation can also be applied to the contrast media 105. The system 100 may be configured to allow the user to select a set concentration of contrast media 105, and the system 100 is configured to dispense the contrast media 105 from the second dispenser 104 and at the same time dispense the delivery media 304 from the second sub-dispenser 308 which is mixed in the mixer 310 to produce the set concentration of contrast media dispensed by the system 100 into the catheter 130.

To use the embolization system 300 in an automatic mode, the user uses the input device 146 to select the desired embolization system 300 operating parameters, such as dispensing flow rate of the first dispenser 102 and/or second dispenser 104, the dispensing pressure of the first sub-dispenser 306, second sub-dispenser 308, and/or second dispenser 104, the on/off cycling of the dispensing of the embolic particle mixture 103 by the first sub-dispenser 306 and second sub-dispenser 308 and/or the dispensing of the contrast media 105 by the second dispenser 104, the cycling of dispensing a bolus of contrast media 105 by the second dispenser 104 and/or dispensing embolic particle mixture 103 (including timing duration and/or cycling rate of on/off cycling of the first dispenser 102 and/or second dispenser 104, the cycle time between dispensing a bolus of contrast media 105 by the second dispenser 104 and a successive bolus of contrast media 105 and/or dispensing embolic particle mixture 103), the size (e.g., volume or time of dispensing) of the bolus of contrast media 105 by the second dispenser 104, the amount of embolic particle mixture 105 to be dispensed in total (by volume, weight or other suitable measure), the total dispensing time for an embolization procedure, etc., or a pre-programmed dispensing program and if desired, setting any one or more of the user selectable characteristics of the dispensing program such as flow rates, pressures, total amount of embolic particle mixture, frequency of injection of contrast media 105, etc.

The user then starts an automated embolization procedure on the embolization system 300 by inputting a “start” command using the input device 146. The controller 136 automatically operates the embolization system 300 to perform the selected automated embolization dispensing program using the first sub-dispenser 306, the second sub-dispenser 308, the second dispenser 104, sensors 134, valves and regulators 148 as controlled by the controller 136. In one generalized automated embolization procedure, the embolization system 300 simultaneously actuates the first sub-dispenser 306 to dispense the embolic particles 302 and the second sub-dispenser 308 to dispense the delivery media 304. The controller 136 controls the relative flow rates of embolic particles 302 dispensed by the first sub-dispenser 306 and the delivery media 304 dispensed by the second dispenser 308 to control the concentration of the embolic particle mixture and produce the concentration selected by the user or the pre-programmed dispensing program. The embolic particles 302 and delivery media 304 flow through the manifold and are mixed in the mixer 310 to produce the selected concentration of embolic particle mixture 103. The embolic particle mixture 310 is injected into the catheter 130 and flows through the catheter 130 and out of the distal end of catheter 130 into the target region of tissue. The flow rate, pressure and/or volume of the dispensed embolic particle mixture 103 is controlled by the controller 136, which may be accomplished using the feedback control process based on the sensor signals from any one or more of the sensors 134a-134e. The embolization system 300 intermittently actuates the second dispenser 104 to dispense a bolus of contrast media 105 into the catheter 130 to inject the contrast media 105 into the target region, which also which may be accomplished using the feedback control process based on the sensor signals from any one or more of the sensors 134a-134e.

Upon injecting the bolus of contrast media 105 from the catheter 130 at the target position, the user can track the contrast media 105 as it is injected into the target region and propagates through the target region of tissue to observe and monitor the progress of embolization of the target region of tissue using a visualization system, such as a fluoroscope or other suitable X-ray system adapted to detect the contrast media and anatomy within the patient and generate real-time images displayed on a monitor.

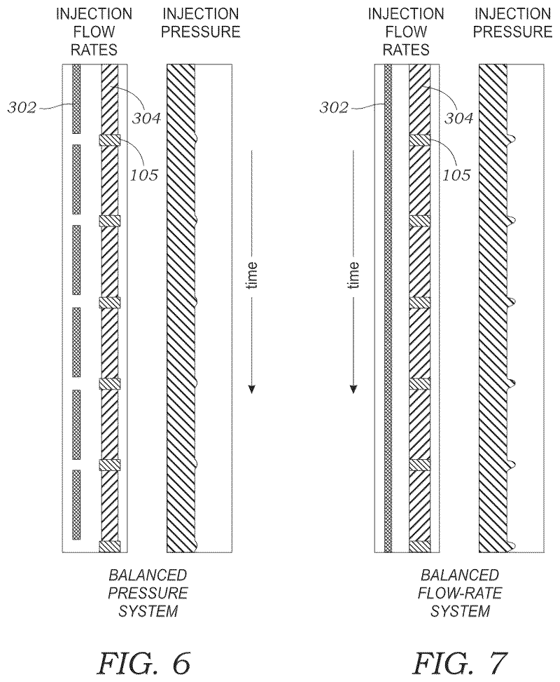

Turning to FIGS. 6 and 7, two different control schemes for using the embolization system 300 to dispense the embolic particle mixture 103 and the contrast media 105 are depicted. The control schemes shown in FIGS. 6 and 7 are similar to the control schemes depicted in FIGS. 2 and 3, except the control schemes in FIGS. 6 and 7 are for the embolization system 300 having separate dispensers for the embolic particles 302 and the delivery media 304. FIG. 6 shows a chart depicting a balanced pressure scheme in which the dispensing/injection pressure is controlled and the flow rate is whatever flow rate stems from controlled dispensing/injection pressure. The balanced pressure scheme can be used to ensure that a safe pressure is always maintained for the fluid being injected into a particular anatomy of the target region. As shown in FIG. 6, in a balanced pressure scheme, the dispensing of the embolic particles 302 by the first sub-dispenser 306 and the delivery media 304 by the second sub-dispenser 308 (i.e., the dispensing of the embolic particle mixture 103) is paused during dispensing of the bolus of contrast media 105 by the second dispenser 104. This maintains a substantially constant injection pressure within the mixer 310, with the potential for only a small increase in pressure during injection of the bolus of contrast media 105. The embolization system 300 may be configured to operate using the balance pressure scheme using the relief valve and/or regulator 148 and/or by configuring the controller 136 to control the dispensing to maintain a set dispensing/injection pressure. For example, relief valve and/or regulator 148 can be set to control the dispensing pressure and/or the controller 136 can control the actuation force of the motorized syringes 108 (in the case that the dispensers 104, 306, 308 are motorized syringes), such as by controlling the current on the motors 134 to obtain the required dispensing pressures by the first sub-dispenser 306, second sub-dispenser 308 and second dispenser 104.

In comparison to the balance pressure scheme, FIG. 7 shows a chart depicting a balanced flow rate scheme in which the dispensing/injection flow rate is controlled and the pressure is whatever pressure stems from the controlled dispensing/injection flow rate. This scheme may be preferred in order to ensure a desired flow rate of embolic particle mixture 103 (or number of embolic particles dispensing rate, which is proportional to the flow rate of embolic particle mixture 103, assuming a relatively constant concentration) during an embolization procedure using the embolization system 300. As shown in FIG. 7, in a balanced flow rate scheme, the dispensing of the embolic particles 302 by the first sub-dispenser 306 is continuous during dispensing of the bolus of contrast media 105 by the second dispenser 104 in order to maintain the set flow rate of embolic particle mixture 103 (the dispensing of delivery media 304 by the second sub-dispenser 308 is paused). As also shown in FIG. 7, this results in an increase in the injection pressure at the output of the manifold 126 during injection of the bolus of contrast media 105. The embolization system 300 can be configured to operate in the balanced flow rate scheme by configuring the controller 136 to control the dispensing flow rate of the first sub-dispenser 306, the second sub-dispenser 308, and the second dispenser 104, such as by controlling the speed at which the respective first and second sub-dispensers 306, 308 and second dispenser 104 are actuated, or otherwise controlling the dispensing flow rates.

The embolization system 300 may also be configured with the dispenser configuration of the embolization system 200 shown in FIG. 4. In such an embolization system, the first and second sub-dispensers 306, 308, and the second dispenser 104 comprise pressurized dispensers same or similar to the pressurized dispensers 200 described herein and shown in FIG. 4. The embolization system also includes pressures sources 204 and the dispensing valves 208 for each of the first and second sub-dispensers 306, 308 and the second dispenser 104. The use and operation of the embolization system 300 having pressurized dispensers is the same as that described for the embolization system 300, except that the dispensing is controlled by controlling the pressurized dispensers 202, instead of the motorized syringes 108. Similarly, the use and operation of the embolization system 300 having pressurized dispensers in performing an embolization procedure to embolize a target region of tissue is the same as for the embolization system 300, except that the dispensing is performed by controllably operating the pressurized dispensers 202.

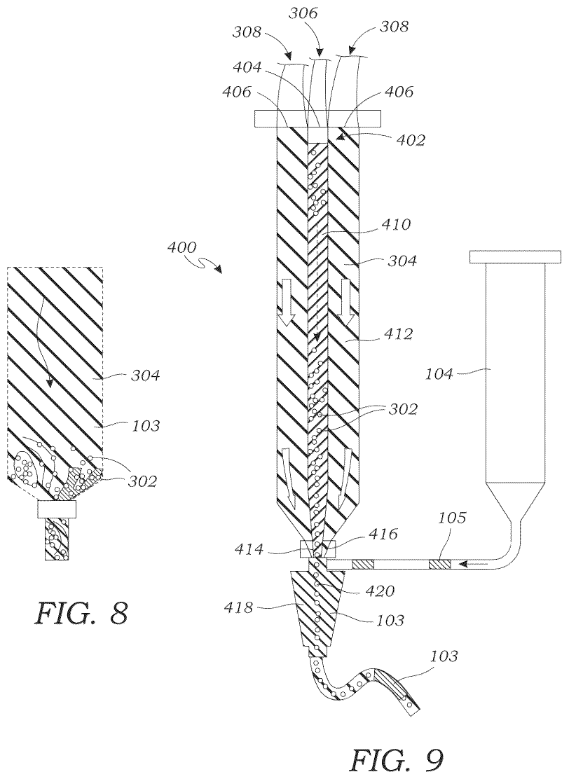

Referring now to FIGS. 8 and 9, a flow rectifier 400 for use with any of the embolization systems described herein, including any of the embolization systems 100, 200, 300, and disclosed variants, will now be described. As shown in FIG. 8, the embolic particles 302 in an embolic particle mixture 103 can aggregate and clog at physical obstructions and obstacles in the flow path, such as at a tapering flow outlet, a junction of a blood vessel, or the like. This aggregation and clogging can also decrease the effectiveness of embolization because the disordered and jumbled distribution of embolic particles 302 in the embolic particle mixture 103 are prone to aggregating and clogging when encountering obstructions and obstacles.

In order to help prevent such aggregation and clogging, the embolization systems disclosed herein may include a flow rectifier 400 which regulates the flow pattern of the flow of the embolic particles 302 and the delivery media 304 and combines the two flows to “mix” the embolic particles 302 and the delivery media 304 in a regular particular arrangement. As shown in FIG. 9, the flow rectifier 400 has a first fluid pathway 410 (an inner tube 410) in the center of the device and a second fluid pathway 412 (an outer tube 412) having an annular cross-section surrounding the first fluid pathway such that the first fluid pathway 410 and second fluid pathway 412 are coaxial. The first fluid pathway 410 has a first inlet 402 connected to, and in fluid communication with, the outlet of the first sub-dispenser 306. The first fluid pathway 410 has a tapered first outlet 414 which is in the center of a tapered second outlet 416 of the second fluid pathway 412.

The first fluid pathway 410 is configured to rectify the flow of embolic particles 302 as the embolic particles 302 flow through the first fluid pathway 412 to produce a more uniform pattern and consistent flow rate of embolic particles 302 at the first outlet 414. The uniform pattern may be a single file alignment of embolic particles 302, or at least a flow with a very small number of embolic particles 302 across the diameter of the outlet 414, such as less than 5 embolic particles 302, or less than 10 embolic particles, or less than 20 embolic particles.

The second fluid pathway 410 is configured to rectify the flow of the delivery media 304 into a uniform laminar flow pattern at the second outlet 416. Accordingly, when the rectified flow of embolic particles 302 and the rectified flow of delivery media 304 combine at the respective first outlet 414 and second outlet 416 to produce a flow of embolic particle mixture 103 having a sheath flow 420 of delivery media 304 surrounding the uniform and aligned pattern of flow 420 of the embolic particles 302. The formation of the sheath flow 420 surrounding the embolic particles 302 may be formed in a 2-dimensional (2D), 3-dimensional (3D), or cylindrical symmetry format, depending on the configuration of the flow rectifier 400. The combination of a cylindrical shear flow 420 surrounding the center flow of embolic particles flow 420 of embolic particles 302 helps avoid contact of the embolic particles 302 with the surrounding walls, cylinder material, and the catheter with the single file or low particle number profile rectification. This reduces the disturbance that a non-rectified injection of embolic particle mixture 103, thereby preventing or reducing the incidence of clogging of a random bolus formed by circulation and turbulence of the flow. In addition, because of the rectification and alignment of the embolic particles 302 by the flow rectifier 400, passive or active measurement can be applied to predict or count the number of embolic particles 302 being dispensed. This count can be used by the controller 136 to control the dispensing of the embolic particle mixture 103, leading to a more predictable treatment control. The organized/rectified laminar flow not only reduces the disturbance from uncontrolled mixing, but also provides uniform distribution throughout the delivery of embolic particles 302 along the flow field or the pressure gradient.

The flow rectifier 400 is not limited to the example shown in FIG. 9, but may be other suitable types of rectifiers such as rectifiers utilizing microchannels, fluid chips, micro-grooves, or microstructures to provide a uniform distribution of embolic particles 302 within a flow of dispensed embolic particle mixture 103. Fluid chips or fluid microchannels can also be used for mixing, prepping, the embolic particles 302, embolic particle mixture 103, and/or contrast media 105 before filling the dispensers of the embolization system and connecting the embolization system to a catheter for use in an embolization procedure.

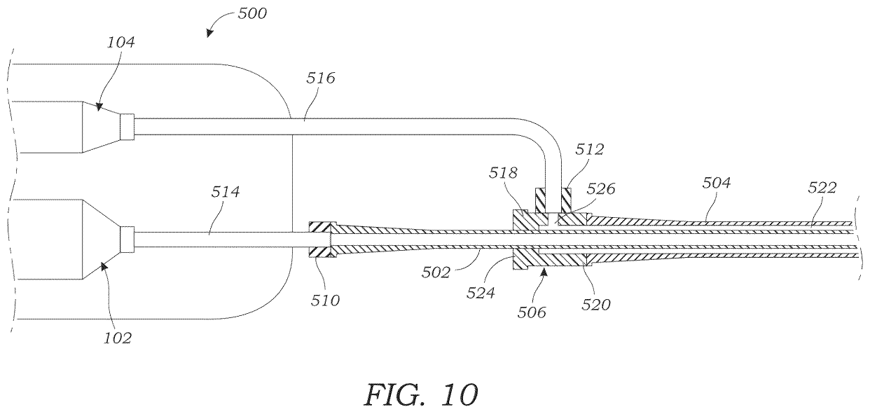

Turning now to FIG. 10, a distal end of an embolization system 500 having different catheters for dispensing the embolic particle mixture 103 and the contrast media 105 is illustrated. The embolization system 500 may be configured like any of the embolization systems 100, 200, 300, and disclosed variants, except that the system 500 has a first catheter 502 for dispensing the embolic particle mixture 103 and a second catheter 504 for dispensing the contrast media 105. The embolic system 500 may also include a catheter hub 506 and first and second fittings 510, 512 for coupling and sealing the first catheter 502 and second catheter 504 to the first and second dispensers 120, 104 in a coaxial configuration. For example, the catheter hub 506 may be a rotating hemostatic valve (RHV) or Touhy-Borst connector. In the illustrated embodiment, the first catheter 502 may be a microcatheter and the second catheter 504 may be a guide catheter which are configured such that the first catheter 502 is insertable into the lumen of the second catheter 504 such that the first catheter 502 and second catheter 504 are coaxial. The first fitting 510 is disposed on a distal end of a first fluid conduit 514 which is in fluid communication with the first dispenser 102. The first fitting 510 is configured to attach to the proximal end of the first catheter 502. The second fitting 512 is disposed on the distal end of a second fluid conduit 516 which is in fluid communication with the second dispenser. The second fitting 512 is configured to attach to a first coupling 518 of the catheter hub 506. The catheter hub 506 has a second coupling 520 configured to be attached to a proximal end of the second catheter 504. The catheter hub 506 also has a compression fitting 524 configured to receive the first catheter 502 to form a fluid tight seal around the first catheter 502. The first catheter 502 inserts through the compression fitting 524 and inserts into the lumen of the second catheter 504, thereby forming an annular flow path 522 between the first catheter 502 and the second catheter 504. The catheter hub 506 has a fluid flow path 526 which fluidly connects the second fluid conduit 516 to the annular flow path 522. The first catheter 502 is connected to, and in fluid communication with the first dispenser and the second catheter 504 is connected to, and in fluid communication with the second dispenser 102 via the annular flow path 522. Accordingly, the first dispenser 102 can dispense embolic particle mixture 103 through the lumen of the first catheter 502, and the second dispenser 104 can dispense contrast media through the annular flow path 522 to respective outlets at a distal end of the respective first and second catheters 502, 504. The embolization system 500 may have any of the features and aspects described herein for the embolization system 100, 200, 300.

The use and operation of the embolization system 500 in performing an embolization procedure to embolize a target region of tissue is also substantially the same as for the respective embolization systems 100, 200 and 300, except for the placement and function of the first catheter 502 and second catheter 504. In one method, the second catheter 504 is used like a guide catheter 504 and is inserted and advanced through a vascular system to a target position proximate a target region of tissue to be embolized, as described herein. The second catheter 504 is connected to the catheter hub 506 by connecting the proximal end of the second catheter 504 to the second coupling 520 of the catheter hub 506. The first catheter 502 is then inserted into the lumen of the second catheter 504 via the compression fitting 524 of the catheter hub 506 and is advanced through the lumen of the second catheter 504 to the target position. Alternatively, the first catheter 502 can be first inserted and advanced to the target position, and then used similar to a guidewire. The second catheter 504 is then advanced over the first catheter 502 with the first catheter 502 within the lumen of the second catheter 504. The proximal end of the second catheter 504 is connected to the second coupling 520 of the catheter hub 506 before or after advancing the second catheter 504 over the first catheter 502 to the target position. The first fitting 510 is connected to the proximal end of the first catheter 502 and the second fitting 512 is connected to the first coupling of the catheter hub 506 to connect the embolization system 500 to the first and second catheters 502, 504.

The embolization system 500 is then operated as described above for the respective embolization systems 100, 200 and 300, in a manual mode, automatic mode, or any other suitable manner.

Various aspects of the disclosure are described hereinafter with reference to the figures. It should be noted that the figures are not drawn to scale and that elements of similar structures or functions are represented by like reference numerals throughout the figures. It should also be noted that the figures are only intended to facilitate the description, and are not intended as an exhaustive description of the disclosure or as a limitation on the scope of the disclosure, which is defined only by the appended claims and their equivalents. In addition, the respective illustrated aspects need not each have all the features or advantages of aspects described herein. A feature or an advantage described in conjunction with a particular aspect of the disclosure is not necessarily limited to that aspect and can be practiced with any other aspect even if not so illustrated.

What is claimed is:

1. A system for dispensing embolic particles into a catheter for delivery into a patient to embolize a target region, comprising:
a first dispenser for dispensing an embolic particle mixture into a catheter, the embolic particle mixture comprising a mixture of embolic particles and delivery media, the first dispenser electronically controllable to automatically control dispensing of the embolic particle mixture;
a second dispenser for dispensing a contrast media into a catheter, the second dispenser electronically controllable to automatically control dispensing of the contrast media; and
a controller operably coupled to the first dispenser and the second dispenser, the controller configured to automatically control the first dispenser and the second dispenser to controllably dispense the embolic mixture from the first dispenser and controllably dispense the contrast media from the second dispenser.

2. The system of claim 1, wherein the first dispenser comprises a first powered injector configured to controllably dispense a pressurized flow of the embolic particle mixture from the first dispenser, and the second dispenser comprises a second powered injector configured to controllably dispense a pressurized flow of the contrast media from the second dispenser.

3. The system of claim 2, wherein the first dispenser comprises a first powered syringe and the second powered injector comprises a second powered syringe.

4. The system of claim 1, wherein the first dispenser comprises a first sub-dispenser for dispensing the embolic particles into a mixer and a second sub-dispenser for dispensing the delivery media into the mixer; and
the system further comprises the mixer in fluid communication with the first sub-dispenser and the second sub-dispenser, the mixer configured to mix the embolic particles and the delivery media and output the embolic particle mixture.

5. The system of claim 4, wherein the first sub-dispenser comprises a first powered injector configured to controllably dispense a pressurized flow of embolic particles into the mixer, the second sub-dispenser comprises a second powered injector configured to controllably dispense a pressurized flow of delivery media into the mixer, and the second dispenser comprises a third motorized injector configured to controllably dispense a pressurized flow of the contrast media from the second dispenser.

6. The system of 1, wherein the controller is selectably programmable to control a characteristic of one of dispensing of the embolic particle mixture by the first dispenser and dispensing of the contrast media by the second dispenser.

7. The system of claim 6, wherein the characteristic of dispensing by the first dispenser and second dispenser is at least one of a flow rate of dispensing, a pressure of dispensing, and a volume of dispensing.

8. The system of claim 6, further comprising a first sensor operably connected to the controller and configured to detect an attribute of the dispensing by at least one of the first dispenser and second dispenser and to output a first sensor signal related to the attribute, wherein the controller is configured to use a feedback control process utilizing the first sensor signal to control the dispensing by at least one of the first dispenser and second dispenser.

9. The system of claim 8,
wherein the first sensor comprises one of a flow rate sensor, a pressure sensor, a force sensor, torque sensor, and electrical current sensor,
wherein the attribute of dispensing is one of an injection flow rate and an injection pressure of the dispensing by at least one of the first dispenser and second dispenser into the catheter, and the controller controls at least one of the first dispenser and second dispenser using the feedback control process to achieve a prescribed characteristic of dispensing into the catheter, and
wherein the prescribed characteristic of dispensing into the catheter includes one or more of flowrate, pressure and concentration of dispensed embolic particle mixture or contrast media.

10. The system of claim 1, wherein the controller is configured to automatically perform a process comprising
dispensing an embolic particle mixture at a prescribed flow rate controlled by the controller; and
intermittently dispensing a bolus of contrast media at a prescribed flow rate controlled by the controller.

11. The system of claim 1, further comprising a flow rectifier having an inlet connected to, and in fluid communication with, the outlet of the manifold and a rectifier outlet, the flow rectifier configured to regulate the flow pattern of the embolic particle mixture as it flows through the rectifier to produce a more uniform flow of the embolic particles withing the delivery media.

12. The system of claim 11, wherein the rectifier is configured to produce a laminar flow of the embolic particle mixture, wherein the embolic particles are aligned proximate the center line of the flow the embolic particle mixture at the outlet of the rectifier.

13. The system of claim 1, further comprising a manifold in fluid communication with both the first dispenser and the second dispenser such that the first dispenser and second dispenser dispense into the manifold, the manifold having an outlet for dispensing out of the manifold.

14. The system of claim 1, further comprising
a first catheter in fluid communication with the first dispenser; and
a second catheter in fluid communication with the second dispenser,
wherein the first catheter is insertable into a lumen of the second catheter such that the first dispenser is configured to dispense embolic particle mixture into the first catheter and the second catheter is configured to dispense contrast media into an annular flow path formed between the first catheter and the second catheter.

15. The system of claim 14, further comprising
a first fitting for connecting to a proximal end of the first catheter to fluidly connect the first dispenser to the first catheter; and
a second fitting for connecting to a catheter hub to fluidly connect the second dispenser to the annular flow path,
wherein the catheter hub comprises a first coupling for connecting to the second fitting, a second coupling for connecting to a proximal end of the second catheter, and a compression fitting for receiving the first catheter and forming a fluid tight seal around the first catheter, and
wherein the catheter hub comprises a fluid flow path fluidly connecting the first coupling to the annular flow path.

16. The system of claim 1, wherein the embolic particle mixture has a low concentration of embolic particles to embolic particle mixture in a range from 2.0-0.05% by volume.

17. The system of claim 1, wherein the controller is configured to be triggered to actuate the second dispenser to dispense the contrast media by a manual input by a user.

18. The system of claim 1, wherein the controller is configured to automatically initiate dispensing of the contrast media by the second dispenser.
